SEQUENCE LISTING (46 sequences)

SEQ ID NO: 1 - protein, 8 aa, Artificial Sequence
  affinity-selected peptide
VSPPPQHS

SEQ ID NO: 2 - protein, 8 aa, Artificial Sequence
  affinity-selected peptide
VSPQSAPP

SEQ ID NO: 3 - protein, 8 aa, Artificial Sequence
  affinity-selected peptide
VTPPSQHA

SEQ ID NO: 4 - protein, 8 aa, Artificial Sequence
  affinity-selected peptide
VTPPTQHQ

SEQ ID NO: 5 - protein, 8 aa, Artificial Sequence
  affinity-selected peptide
DLTSNQAT

SEQ ID NO: 6 - protein, 8 aa, Artificial Sequence
  affinity-selected peptide
DRTSNQAT

SEQ ID NO: 7 - protein, 8 aa, Artificial Sequence
  affinity-selected peptide
ERPPNPSS

SEQ ID NO: 8 - protein, 8 aa, Artificial Sequence
  affinity-selected peptide
ERSSQANM

SEQ ID NO: 9 - protein, 8 aa, Artificial Sequence
  affinity-selected peptide
ERTTSAHT

SEQ ID NO: 10 - protein, 8 aa, Artificial Sequence
  affinity-selected peptide
VSPPSNPS

SEQ ID NO: 11 - protein, 8 aa, Artificial Sequence
  affinity-selected peptide
VTPPSQHA

SEQ ID NO: 12 - protein, 8 aa, Artificial Sequence
  affinity-selected peptide
DPKSPLHT

SEQ ID NO: 13 - protein, 8 aa, Artificial Sequence
  affinity-selected peptide
DPRPAQHT

SEQ ID NO: 14 - protein, 8 aa, Artificial Sequence
  affinity-selected peptide
DPRSPASL

SEQ ID NO: 15 - protein, 8 aa, Artificial Sequence
  affinity-selected peptide
EPRLAHGA

SEQ ID NO: 16 - protein, 8 aa, Artificial Sequence
  affinity-selected peptide
TPGQDKAQ

SEQ ID NO: 17 - protein, 8 aa, Artificial Sequence
  affinity-selected peptide
VPPPGQHQ

SEQ ID NO: 18 - protein, 8 aa, Artificial Sequence
  affinity-selected peptide
VPPPSASS

SEQ ID NO: 19 - protein, 8 aa, Artificial Sequence
  affinity-selected peptide
VPPPSNPS

SEQ ID NO: 20 - protein, 8 aa, Artificial Sequence
  affinity-selected peptide
VPPPSPHS

SEQ ID NO: 21 - protein, 8 aa, Artificial Sequence
  affinity-selected peptide
VPPPSPQS

SEQ ID NO: 22 - protein, 8 aa, Artificial Sequence
  affinity-selected peptide
VPPPSQSQ

SEQ ID NO: 23 - protein, 8 aa, Artificial Sequence
  affinity-selected peptide
VPPSSSSP

SEQ ID NO: 24 - protein, 8 aa, Artificial Sequence
  affinity-selected peptide
VPQQNKAQ

SEQ ID NO: 25 - protein, 8 aa, Artificial Sequence
  affinity-selected peptide
VSTQSTHP

SEQ ID NO: 26 - protein, 8 aa, Artificial Sequence
  affinity-selected peptide
VTPPQSSS

SEQ ID NO: 27 - protein, 8 aa, Artificial Sequence
  affinity-selected peptide
VTPPTSPQ

SEQ ID NO: 28 - protein, 8 aa, Artificial Sequence
  affinity-selected peptide
VTPQGSHP

SEQ ID NO: 29 - protein, 8 aa, Artificial Sequence
  affinity-selected peptide
VTPSSPHS

SEQ ID NO: 30 - protein, 8 aa, Artificial Sequence
  affinity-selected peptide
DNKMTSQS

SEQ ID NO: 31 - protein, 8 aa, Artificial Sequence
  affinity-selected peptide
DPHLAGGL

SEQ ID NO: 32 - protein, 8 aa, Artificial Sequence
  affinity-selected peptide
DPKGPHSM

SEQ ID NO: 33 - protein, 8 aa, Artificial Sequence
  affinity-selected peptide
DPKSPQQT

SEQ ID NO: 34 - protein, 8 aa, Artificial Sequence
  affinity-selected peptide
DPNKSHQS

SEQ ID NO: 35 - protein, 8 aa, Artificial Sequence
  affinity-selected peptide
DPSKRTQP

SEQ ID NO: 36 - protein, 8 aa, Artificial Sequence
  affinity-selected peptide
DRPSPNTV

SEQ ID NO: 37 - protein, 8 aa, Artificial Sequence
  affinity-selected peptide
EPHRAASV

SEQ ID NO: 38 - protein, 8 aa, Artificial Sequence
  affinity-selected peptide
EPNKHSQS

SEQ ID NO: 39 - protein, 8 aa, Artificial Sequence
  affinity-selected peptide
VTPPQQGS

SEQ ID NO: 40 - protein, 8 aa, Artificial Sequence
  affinity-selected peptide
DRSPSSPT

SEQ ID NO: 41 - protein, 8 aa, Artificial Sequence
  affinity-selected peptide
VPIPYNGE

SEQ ID NO: 42 - protein, 8 aa, Artificial Sequence
  affinity-selected peptide
VSSNQAPP

SEQ ID NO: 43 - protein, 8 aa, Artificial Sequence
  Motif I from affinity-selected peptides
VXPXXXXX

SEQ ID NO: 44 - protein, 8 aa, Artificial Sequence
  Motif II from affinity-selected peptides
DPXXXXXX

SEQ ID NO: 45 - protein, 8 aa, Artificial Sequence
  Motif III from affinity-selected peptides
XRXXXXXX

SEQ ID NO: 46 - protein, 8 aa, Artificial Sequence
  Motif IV from affinity-selected peptides
DLTSNQAT

(19) United States
(12) Patent Application Publication
(10) Pub. No.: US 2005/0137136 A1
(43) Pub. Date: Jun. 23, 2005

(54) Biospecific probes against Salmonella

(75) Inventors: Valery A. Petrenko, Auburn, AL (US); Iryna B. Sorokulova, Auburn, AL (US); Bryan A. Chin, Auburn, AL (US); James M. Barbaree, Dadeville, AL (US); Vitaly J. Vodyanoy, Auburn, AL (US); I-Hsuan Chen, Auburn, AL (US); Alexandre M. Samoylov, Auburn, AL (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Auburn University, Auburn, AL (US)

(21) Appl. No.: 10/835,405

(22) Filed: Apr. 29, 2004

Related U.S. Application Data
(60) Provisional application No. 60/466,485, filed on Apr. 29, 2003.

(51) Int. Cl. A61K 38/08; C07K 7/06; C07H 21/04

(52) U.S. Cl. 514/16; 530/329; 536/23.1; 435/69.1; 435/320.1; 435/325

(57) ABSTRACT

Compositions and methods for binding to Salmonella bacteria are provided. The compositions comprise peptide sequences which bind to Salmonella bacteria with high specificity. The compositions are useful in identification, detection, and isolation of Salmonella bacteria. The compositions are also useful for the delivery of a wide variety of compounds to Salmonella bacteria or their vicinity. Such compounds include nucleotides, proteins (including, for example, toxins), liposomes, and small molecule pharmaceuticals. The compositions are also useful in identifying bacterial cell surface markers to which the compositions bind.

### CROSS-REFERENCE TO RELATED APPLICATIONS

This application claims the benefit of U.S. Provisional Application No. 60/466,485, filed Apr. 29, 2003.

### FEDERAL FUNDING DISCLOSURE

This work was partially supported by U.S. Army Grant (ARO/DARPA) # DAAD19-01-10454, U.S. Army Grant (ARO) # DAAG55-98-1-0258, and a USDA RFID grant.

### FIELD OF THE INVENTION

The invention relates to identification of peptides for the recognition, isolation, characterization, and targeting of Salmonella.

### BACKGROUND OF THE INVENTION

Salmonella is a group of bacteria that can cause diarrheal illness in animals, including humans. Salmonella infections can be caused by any of more than 2,000 strains of bacteria known as Salmonella. Among Salmonella, the bacteria known as Salmonella enteritidis and Salmonella typhimurium are responsible for causing the majority of Salmonella infections. According to the United States Centers for Disease Control and Prevention (“CDC”), every year, approximately 800,000 to 4 million cases of Salmonella result in 500 deaths in the United States. Symptoms of Salmonella infection include diarrhea, fever, and abdominal cramps which develop 12 to 72 hours after infection. The illness usually lasts 4 to 7 days.

Typical Salmonella infections involve enterocolitis, or an infection in the lining of the small intestine. While most human patients recover from Salmonella infections without treatment, infections are most likely to be severe among young children, the elderly, and the immunocompromised. In a severe infection, a patient can require medical treatment for dehydration and the infection can spread from the intestines, possibly causing life-threatening meningitis and septicemia.

Foods contaminated with Salmonella usually look and smell normal. Contaminated foods often include beef, poultry, milk, and eggs, but all foods, including vegetables, may become contaminated. There is no vaccine to prevent Salmonella infection, and some Salmonella bacteria have become resistant to antibiotics, largely as a result of the use of antibiotics to promote the growth of feed animals. For these reasons, Salmonella is a potential bioterror agent that could be used to contaminate the food supply. In fact, Salmonella was used by the Rajneeshee religious cult in Oregon to contaminate salad bars at local restaurants, resulting in 751 cases of Salmonella infections. This outbreak was a trial run of the cult's plan to infect residents on election day in order to influence the results of county elections.

The identification of Salmonella typhimurium in food products takes up to 48 hours using laboratory-based culturing and testing. To date, most analytical platforms for rapid detection of Salmonella and other threat agents exploit antibodies that bind the agent and generate a measurable signal. However, the applicability of antibodies as probes for field monitoring is hindered by their sensitivity to unfavorable environmental conditions. Other methods involve the use of PCR, which is a relatively time-consuming and typically labor-intensive method of screening multiple samples.

Thus, there remains an urgent need for methods of rapid detection of Salmonella to prevent its distribution through the food delivery chain. There also remains a need for a stable, reproducible, and inexpensive alternative to antibodies for use as a molecular recognition probe.

### SUMMARY OF THE INVENTION

Compositions and methods for binding to Salmonella bacteria are provided. The compositions comprise peptide sequences which bind to Salmonella bacteria with high specificity.

The compositions are useful in identification, detection, and isolation of Salmonella bacteria. The compositions are also useful for the delivery of a wide variety of compounds to Salmonella bacteria or their vicinity. Such compounds include nucleotides, proteins (including, for example, toxins), liposomes, and small molecule pharmaceuticals. The compositions are also useful in identifying bacterial cell surface markers to which the compositions bind.

### BRIEF DESCRIPTION OF THE DRAWINGS

FIG. 1 shows binding of different phages from Selection 1 (see Example 4) to Salmonella cells. Seven selected phages and one control phage were assayed for binding to Salmonella. The control phage f8-5 is designated as “8,” while the affinity-selected phages are numbered 1-7. The foreign peptides displayed by the phage were as follows: 1, VTPPSQHA; 2, VTPPTQHQ; 3, VSPPPQHS; 4, VSPQSAPP; 5, ERPPNPSS; 6, VSPPSNPS; 7, ERPPNPSS. The vertical axis shows the ELISA signal obtained for each binding assay (in mOD/ml).

FIG. 2 shows binding of Salmonella cells to different phages from Selection 1 (see Example 4). Seven selected phages and one control phage were assayed for their ability to bind Salmonella. The control phage f8-5 is designated as “8,” while the affinity-selected phages are numbered 1-7. The foreign peptides displayed by the phage were as follows: 1, VTPPSQHA; 2, VTPPTQHQ; 3, VSPPPQHS; 4, VSPQSAPP; 5, ERPPNPSS; 6, VSPPSNPS; 7, ERPPNPSS. The vertical axis shows the ELISA signal obtained for each binding assay (in mOD/ml).

FIG. 3 shows the results of a coprecipitation test for binding of phage to Salmonella. Coprecipitation was tested as in Example 4, but using individual clones instead of a whole library. The percentage yield is shown on the horizontal axis for selected phage, which are arrayed on the vertical axis. The foreign peptides displayed by the phage are as follows: 1, f8-5 vector; 2, VSPPPQHS; 3, VTPPSQHA; 4, VTPPQSSS; 5, VPQQDKAQ; 6, VNYDDMTST; 7, DRSPSSPT; 8, VSSNQAPP; 9, VSPPSNPS; 10, DLTSNQAT; 11, DRPSPNTV; 12, VSPQSAPP; 13, ERPPNPSS; 14, VTPPTQHQ.

FIG. 4 shows the results of a binding assay in which fluorescently-labeled phage displaying the foreign peptide VTPPTQHQ were incubated with Salmonella adsorbed to a plastic surface. After 1 hour of incubation, unbound phage were removed by washing with TBS/0.5% Tween™. Salmonella were then analyzed by Fluorescence-Activated Cell Sorting (FACS). Fluorescence values are arrayed on the horizontal axis, while the number of cells at each fluorescence value is arrayed on the vertical axis.

### DETAILED DESCRIPTION OF THE INVENTION

Targeting of Salmonella cells for diagnostic, prognostic, and treatment purposes all require targeting specificity. The present invention provides compositions that can be used to characterize a particular cell population as well as to deliver compounds to that cell population. Delivery may occur by bringing a compound into the vicinity of the target cells, such as to the cell surface, or delivery may capitalize on endogenous cellular pathways of macromolecular transport such that compounds are internalized within the target cells. In this manner, delivery of compounds may be accomplished via the receptor-mediated endocytosis pathway employing molecular conjugate vectors.

The invention is drawn to peptides that have been shown to bind Salmonella or to bind to Salmonella with high specificity. Further provided are nucleic acids comprising nucleotide sequences that encode the peptides of the invention, and vectors comprising these nucleic acids. The peptides are useful for targeting compounds to Salmonella cells for diagnostic, prognostic, and/or therapeutic purposes. Such compounds include labeling compounds used for cytology or histology, pharmaceuticals, proteins (including, for example, toxins), liposomes, and genetic material such as, for example, DNA. In this manner, the peptides of the invention may be used to effect gene transfer into target cells in vivo and may also be used with tissue samples in vitro and/or in situ for diagnostic and/or prognostic purposes. The peptides of the invention may also be used alone or as displayed on phage to create biosensor devices for the detection of Salmonella. See, e.g., copending application Ser. No. 10/068,570, filed Feb. 6, 2002, and copending application Ser. No. 10/289,725, filed Nov. 7, 2002.

While the peptides of the invention have been selected based on their ability to bind Salmonella cells, it is understood that these peptide sequences may also bind to other cells that are not Salmonella cells, such as, for example, E. coli. In this manner, the peptides of the invention may also be useful in diagnosis and/or therapy of various bacterial infections, or may be useful in comparing various cell populations based on cell surface marker characteristics.

The peptides of the invention are generally short peptide ligands, and are referred to herein as “synthetic peptides” or “peptides.” Synthetic peptides that have been produced as a fusion protein with phage coat proteins are also referred to herein as “foreign peptides,” because the synthetic peptide is generally foreign to the phage or is found in a non-native context. The synthetic peptides of the invention may exhibit at least two-fold, three-fold, four-fold, five-fold, six-fold, seven-fold, ten-fold, twenty-fold, thirty-fold, forty-fold, fifty-fold, sixty-fold, seventy-fold, one hundred-fold, one thousand-fold, ten thousand fold, one hundred thousand-fold, one million-fold, or more increased binding affinity for Salmonella cells relative to at least one category or type of other cells, or relative to the binding exhibited by a control phage which does not express the synthetic peptide. Synthetic peptides that exhibit such binding characteristics are said to exhibit preferential binding to Salmonella cells. Synthetic peptides that do not exhibit at least a two-fold increased binding affinity for Salmonella cells relative to another category or type of other cells but that bind to Salmonella cells are simply said to bind to Salmonella cells.

The synthetic peptides of the invention are cell-binding and cell-entry peptides. For the most part, these synthetic peptides will comprise at least about 5 to about 50 amino acids, preferably at least about 5 to about 30 amino acids, more preferably at least about 6, 7, 8, 9, 10, 11, 12, 13, 14, 15, 16, 17, 18, 19, or up to about 20 amino acids. It is recognized that motifs or sequence patterns may be identified among the peptides that are capable of binding to a target. Such motifs identify key amino acids or patterns of amino acids that are essential for binding. Motifs may be determined from an analysis of peptide patterns that are capable of binding Salmonella cells. Such motifs may be as short as 3 amino acids in length, or they may be 4, 5, 6, 7, 8, 9, 10, 11, 12, 13, 14, 15, 16, 17, 18, 19, or 20 amino acids in length. Motifs can contain amino acid residues which are invariant as well as amino acid residues which may be substituted by one or more other amino acids without affecting the properties conferred by the motif. For example, motif I (SEQ ID NO:43) contains invariant amino acids as the first (V) and third (P) amino acids in the motif, while the amino acids at positions 2, 4, 5, 6, 7, and 8 may be one of several amino acids. Thus, sequences containing this motif include, for example, VPPNPAHA and VSPPQNHP.

Using the selection schemes described herein, four motifs were identified in affinity-selected peptides, as follows:

TABLE 1
Motifs identified in affinity-selected peptides

| | Position in peptide | | | | | | | |
|---|---|---|---|---|---|---|---|---|
| Motif | 1 | 2 | 3 | 4 | 5 | 6 | 7 | 8 |
| I | V | P/S/T | P | N/P/Q | P/Q/S/T | A/N/Q/S | H/P/S | A/P/Q/S |
| II | D | P | H/K/R | G/L/P/S | A/P | A/G/H/L/Q | G/H/Q/S | L/M/T |
| III | D/E | R | P/S/T | P/S/T | P/S | A/N/S | H/P/T | T/V |
| IV | D | L | T | S | N | Q | A | T |

These motifs are included in the sequence listing as follows: Motif I is set forth in SEQ ID NO: 43; Motif II is set forth in SEQ ID NO:44; Motif III is set forth in SEQ ID NO:45; and Motif IV is set forth in SEQ ID NO:46.

Once identified, these motifs are useful in constructing other peptides for use in targeting cell populations of interest. Motifs may be evaluated by constructing peptides containing the motif and determining the effect of the motif on the peptide's binding properties to populations or cells of interest as compared to control populations or cells (i.e., populations or cells not of interest). It will be appreciated that the creation of variant sequences by the addition of extra copies of the same motif and/or different motifs and/or the addition of other flanking amino acids may enhance the binding properties conferred by a motif on a peptide comprising it. By “enhancing the binding properties” is intended that a peptide shows or confers on an associated compound an increase in desired binding properties or a decrease in undesired binding properties. One of skill in the art can determine appropriate desired or undesired binding properties based on the particular application. For example, a peptide with enhanced binding properties may show increased binding to Salmonella cells compared to another particular cell type, or it may show increased binding to all cell types tested.

One of skill in the art is familiar with techniques to make and test such peptides, for example, as taught herein with appropriate modifications which would be routine to those of skill in the art. Such variant peptides are encompassed by the term “peptide” and “synthetic peptide” as used herein. The synthetic peptides can be classified into linear, cyclic and conformational types. While the invention is not bound by any particular mode of action, it is postulated that shorter peptides, which are generally from about 7 to about 20 amino acids, are involved in linear binding to the target cells. Longer peptides are thought to assume conformational folding and are involved in conformational binding. Cyclic peptide structures can also be constructed for use in the invention. In this manner, a core peptide region such as a motif sequence may be flanked with identical sequences to form cyclic peptides. For such construction, libraries are available commercially. See, for example, the Ph.D.™ phage display peptide library kits from New England Biolabs, Inc. See also, Parmley et al. (1988) Gene 73:305-318; Cortese et al. (1995) Curr. Opin. Biotechnol 6:73-80; Noren (1996) NEB Transcript 8(1):1-5; and Devlin et al. (1990) Science 249:404-406.

While the synthetic peptides of the invention were isolated based on a screen for their ability to preferentially bind Salmonella cells, it is expected that these peptides will also preferentially bind to other bacterial cells and to other cell types, such as, for example, human tissue cells such as liver cells. Peptides may also bind to particular cell types, such as cell types of a particular origin. Thus, the peptides of the invention find use where they preferentially bind at least one target cell population when compared to their binding of at least one non-target-cell population, as can be readily selected by one of skill in the art. Peptides may also bind generally to most cell types; such peptides find use, for example, in applications such as the isolation and identification of cell surface markers and in characterization of the cell surface markers of particular cell populations.

Peptides of the invention were identified and isolated using phage display libraries in particular screening procedures which are more particularly described in the Experimental Examples. Briefly, phage display libraries were created that express random synthetic peptides on the surface of each phage. Thus, the binding properties of each phage in the library are expected to reflect the binding properties of the foreign or synthetic peptide expressed on the phage surface. These phage libraries were then screened for phage having an ability to bind to Salmonella cells where the Salmonella were either adsorbed to a surface, such as a plastic Petri dish, or where the Salmonella cells were in solution. Phage selected in these screens as binding to Salmonella cells were phage that either became associated with the cells or internalized within the cells; these phage are expected to be recovered from the elution buffer fraction or the lysis buffer fraction, respectively, although different reasons may exist for the presence of a particular phage in a particular buffer fraction.

After multiple rounds of selection, the affinity-selected phage were recovered and individually isolated. The nucleotide sequence encoding the synthetic peptide for each phage clone was determined. Individual phage clones were then further assayed to evaluate the binding properties conferred by the synthetic peptide. It is understood that in this context, “synthetic peptide” or “foreign peptide” means a peptide that was introduced into the phage genome by engineering and is not a native phage sequence in its native context. However, because these “synthetic peptides” show binding or preferential binding to Salmonella, it is expected that at least some of the synthetic peptides contain sequences and/or motifs that are found in other proteins or that share similar three-dimensional properties with other proteins, such as, for example, mammalian cell surface proteins.

Thus, as used herein, “synthetic peptide” refers to a peptide which has an amino acid sequence which is not a native sequence or is not in its native context and which displays the ability to bind or preferentially bind to a particular cell population. By “not in its native context” is intended that the peptide is substantially or essentially free of amino acid sequences that naturally flank the amino acid sequence of the peptide in a native protein which comprises the amino acid sequence of the peptide. For example, a synthetic peptide which has the same sequence as a native amino acid sequence may be flanked at either or both ends by 1, 2, 3, 4, 5, 6, 7, 8, 9, 10, 11, 12, 13, 14, 15, 16, 17, 18, 19, 20, 25, 30, 35, 40, 45, or 50 or more amino acids found in the native protein flanking the amino acid sequence that is the same sequence as the synthetic peptide sequence.

The present invention also provides nucleic acids comprising nucleotide sequences that encode these peptides. One of skill in the art, given the amino acid sequence of the peptides of the invention, can readily design and synthesize a nucleic acid comprising a nucleotide sequence that would encode that peptide. Further, because the synthetic peptides of the invention are relatively short, only a relatively small number of nucleotide sequences will encode the particular peptide in question. Further provided by the present invention is the array of peptides described herein which show binding or preferential binding to Salmonella cells. Such an array of binding peptides provides a molecular profile of Salmonella cells and thus serves to further describe and characterize Salmonella cells and Salmonella cell surfaces.

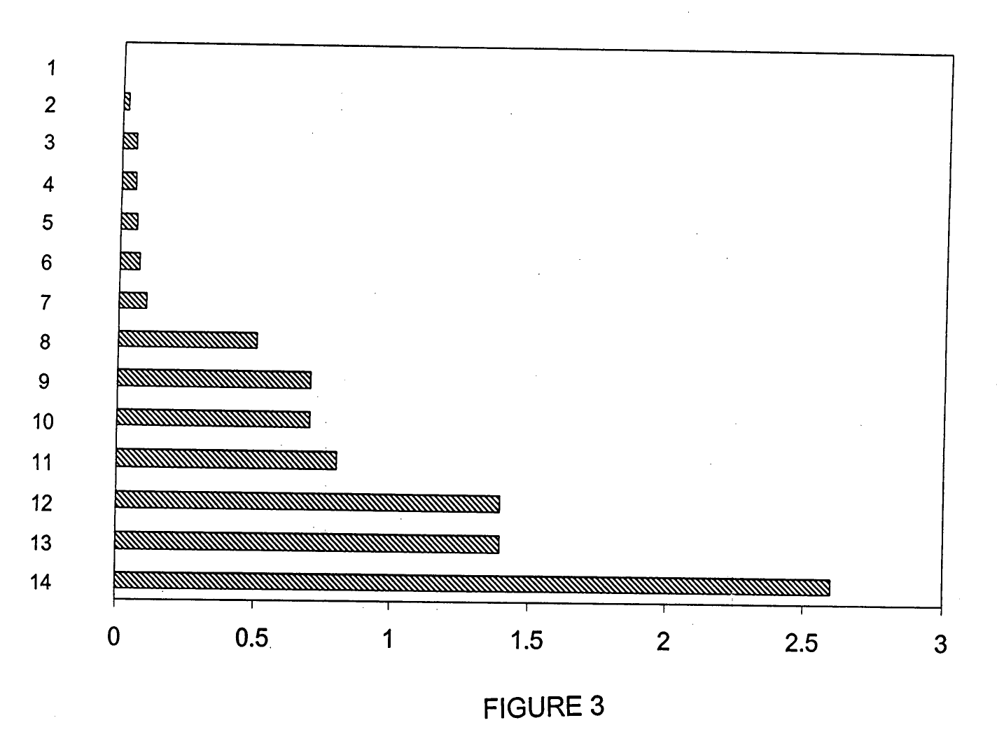

The binding properties of affinity-selected synthetic peptides and the phage expressing them were evaluated. For general methods, see Phage Display: A Laboratory Manual (Cold Spring Harbor Laboratory Press, Cold Spring Harbor, N.Y. (2001)) (e.g., page 26, lines 29-30). Binding specificity of phage was confirmed by counting cell-associated phage following the incubation of individual phage clones with Salmonella cells (see, e.g., Experimental Example 4 and FIG. 3). It was demonstrated that some affinity-selected phage are highly specific and preferentially bind to Salmonella cells by several orders of magnitude compared to control (vector) phage (see, e.g., FIG. 3).

Additional methods are available in the art for the determination of the peptides of the invention. Such methods include selection from a bacteriophage library which expresses random peptides, mirror image phage display to isolate naturally-occurring L-enantiomers in a peptide library, and the like. See, for example, Schumacher et al. (1996) Science 271:1854-1857, herein incorporated by reference. Protocols to select for small peptides that will bind to particular cells have utilized combinatorial library methods such as phage display and one-bead one-compound combinatorial peptide libraries (reviewed by Aina et al. (2002) Biopolymers 66: 184-199). Importantly, the diverse and complex nature of random peptide libraries has the capacity to provide unique peptide sequences for any target receptor molecules, including those that are well-described and those that are previously undetected (Barry et al. (1996) Nat. Med. 2: 299-305). Additionally, the design of phage not only can allow recognition of selective targeting sequences, but also allows rapid isolation of the targeted marker for further characterization. Invented less than 20 years ago (Smith (1985) Science 228: 1315-1317), phage display technology has produced valuable targeting ligands to a variety of cell types, both in vitro and in vivo (Pasqualini and Ruoslahti (1996) Nature 380: 364-366).

Phage display libraries can provide ready sources of small peptides for targeting cell-specific markers. Phage display libraries are heterogenous mixtures of phage clones, each carrying a different foreign or synthetic DNA insert and, therefore, displaying the corresponding individual synthetic peptide on its surface (Smith & Scott (1993) Methods Enzymol. 217: 228-257; Smith & Petrenko (1997) Chem. Rev. 97: 391-410). Bacteriophage libraries can be constructed which display random peptides expressed as fusion proteins with a phage protein. See, Barry et al. (1996) Nature Medicine 2:299-305; Devlin et al. (1990) 249:404-406; Cwirla et al. (1990) Proc. Natl. Acad. Sci. USA 87:6378-6382; and the references cited therein, herein incorporated by reference. Methods for preparing libraries containing diverse populations are also disclosed in Gordon et al. (1994) J. Med. Chem. 37:1385-1401; Ecker and Crooke (1995) BioTechnology 13:351-360; Goodman and Ro, Peptidomimetics For Drug Design, in “Burger's Medicinal Chemistry and Drug Discovery”, Vol. 1, M. E. Wolff (Ed.) John Wiley & Sons 1995, pages 803-861; Blondelle et al. (1995) Trends Anal. Chem. 14:83-92; and Sambrook et al., Molecular Cloning: A Laboratory Manual, Cold Spring Harbor Press, 1989. Each of these references are herein incorporated by reference.

Because such libraries allow functional access to the peptide and provide a physical link be Tween™ the phenotype (displayed peptide) and the genotype (encoding DNA), they lend themselves to a screening process. Clones having desirable binding properties may be separated from non-binding clones by affinity selection. Literally billions of different structures displayed by random phage libraries can be surveyed rapidly and rare binding clones can readily be identified.

It will be appreciated by those of skill in the art that phage display technology as described herein may be adapted in a short period of time for developing phage-derived probes against rare or exotic microbial and viral agents and meet the demand of the biosensor industry for homogenous preparations in virtually unlimited amounts. Furthermore, as opposed to antibodies, the structure of phage is extraordinarily robust, being resistant to heat (such as temperatures of 80° C.), many organic solvents such as acetonitrile, urea in high concentrations (for example, 6M urea), exposure to acid and alkali compounds, and other environmental stresses. Purified phage can be stored indefinitely at moderate temperatures without losing infectivity and probe-binding activity. These characteristics of landscape phage make them superior components of biosensors and other biological detectors.

Different types of phage display libraries exist, varying for example by the size of the synthetic nucleotide insert, gene location of insert, structure of the displayed synthetic peptide, and number of copies of the synthetic peptide expressed on the surface of the phage (Smith & Petrenko (1997) Chem. Rev. 97: 391-410). In phage “landscape libraries,” the synthetic peptides are inserted into the phage major coat protein pVIII (see, e.g., copending application Ser. No. 10/289,725, filed Nov. 7, 2002, and references cited therein, herein incorporated by reference). Each phage virion displays thousands of copies of the synthetic peptide in a repeating pattern, subtending a major fraction of the viral surface. The phage body serves as an interacting scaffold to constrain the synthetic peptide into a particular tertiary conformation, creating a defined “landscape” surface structure that varies from one phage clone to the next. Different phage libraries serve different purposes. For example, because of the large number of synthetic peptide copies displayed on “landscape” phage, these particles can be used effectively to create affinity matrices for isolation and purification of peptide-binding proteins.

Synthetic peptides of the invention can be identified and isolated from phage libraries. The phage are incubated with the cells of interest to select phage that bind to those cells (i.e., phage that become associated with the cell surface or are internalized in those cells). After repeated selection of phage bound to specific cells, phage exhibiting binding or preferential binding to the cells of interest are further characterized by sequencing of the DNA insert encoding the foreign peptide (see, e.g., Experimental Example 1). Phage which have undesirable properties such as binding to the plastic used for cell culture or, e.g., binding to particular populations of non-target cells may be removed by use of a depletion or negative subtraction step (see, e.g., Experimental Example 2).

In order to target compounds to particular cells, it is desirable to identify peptides which bind to cell surface markers on that particular cell or are internalized into that cell. Thus, it is understood in the art that cell surface molecular expression patterns may include receptors which are common to multiple cell lineages or bacterial species, restricted to one or a few cell lineages or bacterial species, and those which are unique to individual cell types or bacterial species. Additionally, among various types of cells, common cell-surface molecules may be expressed similarly or at different densities. To identify synthetic peptides that bind to common cells as well as peptides that bind exclusively to Salmonella cell markers, three selection schemes were used, as described in the Experimental Examples. Each selection scheme yielded a different array of peptides. Confirmation and further characterization of the binding properties of the synthetic peptides was performed using comparative ELISA assays and coprecipitation tests (see, e.g., Experimental Example 4). However, in order to accurately evaluate the affinity of the peptides for the molecules to which they bind (i.e., their ligands), acoustic wave sensor technology (AWST) may also be used to evaluate the interaction of the synthetic peptide with Salmonella cells. Such techniques are known in the art and are also discussed in copending application Ser. No. 10/289,725, filed Nov. 7, 2002, and references cited therein, herein incorporated by reference. Thus, biosensors made using the synthetic peptides of the invention can be used to confirm the specificity of synthetic peptides selected for binding to Salmonella typhimurium versus other cells or other compounds, such as, for example, E. coli. AWST can also be used to evaluate newly-created variant synthetic peptides for their binding affinity to other target or non-target cells. The synthetic peptides of the invention may also be useful as a component of biosensors, for example, biosensors using Acoustic Wave Sensor Technology (AWST).

Once synthetic peptides have been selected for binding affinity to Salmonella, they may be modified by methods known in the art. Such modified peptides and the nucleotide sequences encoding them are referred to herein as “variants,” and are also provided by the present invention. Methods for creating variants include random mutagenesis as well as synthesis of nucleic acids having nucleotide sequences encoding selected amino acid substitutions, deletions, and/or additions. Variant peptides of various lengths and amino acid composition can be constructed and tested for binding affinity and specificity. In this manner, the binding properties of the peptide and the binding properties conferred by the peptide on conjugated compounds may be enhanced. Thus, variant peptides may be created which exhibit specific binding to and/or internalization by other target cells of interest.

Thus, by “variant” or “variant peptide” is intended a peptide that differs by one or more amino acids from a peptide or motif described herein. Variant peptides may be any length and may include multiple copies of motifs or peptide sequences of the invention. “Variants” also encompass peptides having one or more deletions or additions of amino acid residues when compared to a peptide sequence or motif described herein. Thus, a variant may comprise 1, 2, 3, 4, 5, 6, 7, 8, 9, 10, 11, 12, 13, 14, 15, 16, 17, 18, 19, or 20 or more amino acid substitutions, deletions, and/or additions compared to a peptide or motif described herein.

The synthetic peptides of the invention find further use in targeting genes, proteins, pharmaceuticals, antibiotics, cells, and other compounds or entities to Salmonella cells or other cells to which the synthetic peptides bind. In this manner, the peptides can be used with any vector system for delivery of specific nucleic acids or other compositions to the target cells. Here, the term “nucleic acid” is intended to encompass gene sequences, DNA, RNA, and oligonucleotides as well as antisense nucleic acids. Such targeting may be in vitro, in situ, or in vivo. The synthetic peptides find use in in vitro and/or in situ applications such as, for example, diagnosis of Salmonella infection. For diagnostic purposes, the synthetic peptides can be labeled or conjugated with radioisotopes or radionuclides, fluorescent molecules, biotin, enzymes, or any other suitable compound used for localization and/or visualization of particular cell populations.

Another in vitro application for which the peptides of the invention find use is affinity purification of cell surface markers which may be specific to Salmonella cells or to other cells or cell types. In such applications, the synthetic peptides are linked to an appropriate matrix and used to bind the cell surface marker in solution. In this manner, the synthetic peptides are useful in accurate detection of particular cells or cell types in a solution of known or unknown composition. Similarly, the peptides of the invention may be used as components of biosensors for detection and/or characterization of cells and/or cell populations (see, e.g., Experimental Examples 6 and 7). Peptides of the invention may also be used in other areas of health science and engineering for targeting and profiling of associated phenomena, such as infectious diseases, and for the production of bioselective materials and nanomaterials known in the art, such as, for example, biospecific filters, gene- and drug-delivery vehicles, hemostatics, molecular switches, etc.

The nucleotide sequences encoding the synthetic peptides may be incorporated into an expression vector, for example, for production of the synthetic peptide as a fusion protein with another useful protein. Standard techniques for the construction of the vectors of the present invention are well-known to those of ordinary skill in the art and can be found in such references as Sambrook et al. (1989) Molecular Cloning: A Laboratory Manual, 2nd ed. (Cold Spring Harbor, N.Y.). A variety of strategies are available for ligating fragments of DNA, the choice of which depends on the nature of the termini of the DNA fragments; appropriate choices can be readily made by those of skill in the art.

Where the peptides of the invention are targeting a gene for expression in cells of interest, the gene to be expressed will be provided in an expression cassette with the appropriate regulatory elements necessary for expression of the gene. Such regulatory elements are well known in the art and include promoters, terminators, enhancers, etc. The peptides of the invention may also be utilized to target compounds and compositions such as, for example, liposomes, polylysine or other polycation conjugates, and synthetic molecules for delivery to the target cells. See, for example, de Haan et al. (1996) Immunology 89: 488-493; Gorlach et al. (1996) DTWDTsch Tierarytl Wochenschr 103: 312-315; Benameur et al. (1995) J. Phar. Pharmacol. 47: 812-817; Bonanomi et al. (1987) J. Microencapsul 4: 189-200; Zekorn et al. (1995) Transplant Proc. 27: 3362-3363. Thus, the peptides of the invention can be used as free peptides or they can be conjugated or linked to other compounds such as cytotoxic agents including pharmaceutical compounds or other compounds performing useful functions such as, for example, cytotoxic, diagnostic, or delivery functions.

Where desirable, the peptides of the invention may be conjugated or linked to more than one other compound. Examples of antibiotics include amphotericin B, gentamycin sulfate, and pyrimethamine. The synthetic peptides of the invention may be provided as pharmaceutical compositions suitable for parenteral (e.g., subcutaneous, intradermal, intramuscular, intravenous and intraarticular), oral or inhalation administration, or intraocular administration. Alternatively, pharmaceutical compositions of the present invention may be suitable for administration to the mucous membranes of the subject (e.g., intranasal administration).

Pharmaceutical compositions comprise at least one synthetic peptide of the invention and at least one other compound, which may or may not be conjugated to the synthetic peptide. Typically, the other compound is intended to help treat a disease or symptom of a disease; for example, the other compound may be an antibiotic agent intended to kill or inhibit the growth of Salmonella cells. While in some applications the other compound will be conjugated to the synthetic peptide of the invention, in other applications improved results may be obtained where the other compound and the synthetic peptide are not conjugated to each other. Formulations may be conveniently prepared in unit dosage form and may be prepared by any of the methods well-known in the art. Any inert pharmaceutically-acceptable carrier may be used, such as saline, or phosphate-buffered saline, or any such carrier in which the compositions of the present invention have suitable solubility properties for use in the methods of the present invention. Reference is made to Osol, ed. (1980) Remington's Pharmaceutical Sciences (Merck Publishing Company, Easton, Pa.) for methods of formulating pharmaceutical compositions.

The following experiments are offered by way of illustration and not by way of limitation.

### Experimental

All general methods of handling phage display libraries, including phage propagation, purification, titering, production of pure phage clones, and isolation of phage DNA are described in detail in Phage Display: A Laboratory Manual (Cold Spring Harbor Laboratory Press, Cold Spring Harbor, N.Y. (2001)).

### Example 1

### Selection of Phase that Bind to Salmonella Cells

Selection of phages that bind to Salmonella cells was performed using five rounds of selection with an 8-mer library (i.e., a phage display library in which each foreign peptide was 8 amino acids in length). In each round, phage bound to Salmonella typhimurium were recovered consecutively with low-pH elution buffer (to remove cell-surface-bound phage) and with lysis buffer (to recover phage that had been internalized into the Salmonella cells). Elution buffer contains 0.1 N HCl and 1 mg/ml bovine serum albumin (BSA). It is made by mixing water and appropriate amounts of 50 mg/ml BSA and 0.4 N HCl and adjusting the pH to 2.2 with glycine; the solution is then filter-sterilized and stored at room temperature. Lysis buffer contains 2% sodium deoxycholate, 10 mM Tris, and 2 mM EDTA; the pH is adjusted to 8.0 and the solution is ultrafiltrated.

Recovered phages were amplified and used in the next round of selection against Salmonella cells. In each round, the yield of Salmonella-associated phages were determined as a ratio of output phage to input phage. This ratio increased with each successive round of selection, indicating the successful selection of specific phage clones. After the fifth round of selection, individual selected phages were propagated. Phage DNA was isolated and the peptide-encoding regions were sequenced to determine the identity of the foreign peptide displayed by the phage.

TABLE 2

Foreign peptides expressed by phage selected
for ability to bind to Salmonella

| | | |
|---|---|---|
| Peptide sequence | Frequency | SEQ ID NO: |

Isolated from Eluate

| | | |
|---|---|---|
| VSPPPQHS | 1 | 1 |
| VSPQSAPP | 1 | 2 |
| VTPPSQHA | 10 | 3 |
| VTPPTQHQ | 4 | 4 |

Isolated from Lysate

| | | |
|---|---|---|
| DLTSNQAT | 1 | 5 |
| DRTSNQAT | 1 | 6 |
| ERPPNPSS | 8 | 7 |
| ERSSQANM | 1 | 8 |
| ERTTSAHT | 1 | 9 |
| VSPPSNPS | 2 | 10 |
| VTPPSQHA | 1 | 11 |

### Example 2

### Selection of Phage with Enhanced Binding to Salmonella Cells

To isolate phage with stronger and more selective binding properties, the stringency of selection was increased over that used in Example 1. In this selection scheme, the phage library of interest was first exposed to plastic and BSA to eliminate phage that bound to these compositions. In this depletion procedure, a 25 μl library aliquot in 400 μl of buffer (1% BSA, 0.5% Tween™ in TBS) was added to an empty 35-mm Petri dish and incubated for 1 hour at room temperature.

Two phage libraries were used in this selection scheme—an 8-mer library and a 9-mer library. Phage were then selected for binding to Salmonella as in Example 1. To further enhance the selection process, the plate with adsorbed phage was also washed with a detergent solution (0.5% Tween™ in TBS) to better remove nonspecifically-bound phage. As in Example 1, successive rounds of selection provided increased yields of phage in the elution fraction but not in the lysis fraction of phage. After the fourth round of selection, individual phage clones were purified and the region encoding the foreign peptide was sequenced.

TABLE 3

Foreign peptides expressed by phage selected
with revised screening process

| | | |
|---|---|---|
| Peptide sequence | Frequency | SEQ ID NO: |

Isolated from Eluate

| | | |
|---|---|---|
| DPKSPLHT | 1 | 12 |
| DPRPAQHT | 1 | 13 |
| DPRSPASL | 2 | 14 |
| EPRLAHGA | 1 | 15 |
| TPGQDKAQ | 1 | 16 |
| VPPPGQHQ | 1 | 17 |
| VPPPSASS | 1 | 18 |
| VPPPSNPS | 1 | 19 |
| VPPPSPHS | 3 | 20 |
| VPPPSPQS | 2 | 21 |
| VPPPSQSQ | 2 | 22 |
| VPPSSSSP | 1 | 23 |
| VPQQNKAQ | 1 | 24 |
| VSTQSTHP | 1 | 25 |
| VTPPQSSS | 1 | 26 |
| VTPPTSPQ | 1 | 27 |
| VTPQGSHP | 1 | 28 |
| VTPSSPHS | 1 | 29 |

Isolated from Lysate

| | | |
|---|---|---|
| DNKMTSQS | 1 | 30 |
| DPHLAGGL | 1 | 31 |
| DPKGPHSM | 1 | 32 |
| DPKSPQQT | 1 | 33 |
| DPNKSHQS | 1 | 34 |
| DPSKRTQP | 1 | 35 |
| DRPSPNTV | 1 | 36 |
| EPHRAASV | 1 | 37 |
| EPNKHSQS | 1 | 38 |
| VTPPQQGS | 1 | 39 |

### Example 3

### Selection of Phase with Enhanced Binding to Salmonella Cells in Solution

In Examples 1 and 2, phage were selected for their ability to bind to Salmonella cells that were adsorbed onto a plastic surface. In this selection scheme, phage were selected for their ability to bind Salmonella typhimurium cells in solution. Phage libraries were heated and depleted with precipitation to remove self-precipitating phage clones before incubation with the suspension of Salmonella. In the heating step, a portion of 25 μl of a library in 400 μl TBS was heated to 70° C. for 10 minutes. Tween™ detergent was then added to the aliquot to a final concentration of 0.5%, and the suspension was centrifuged for 15 minutes at 13,000 rpm. The supernatant was then mixed with 400 μl of a suspension of Salmonella cells (at a concentration of 0.8 OD measured at 620 nm) in TBS/0.5% Tween™ detergent and incubated for 1 hour at room temperature.

Phage bound to Salmonella were precipitated by centrifugation at 3,500 rpm for 10 minutes and washed 10 times with TBS/0.5% Tween™ detergent followed by recentrifugation. After the final wash, cell-associated phage were eluted from the pellet with 400 μl of elution buffer, and the pellet was again precipitated by centrifugation at 3,500 rpm for 10 minutes. The supernatant was neutralized with 75 μl of 1M Tris at pH 9.1, and the pellet was lysed with 0.25 ml of lysis buffer. Both the elution fraction and the lysis fraction were amplified separately in E. coli strain K91BK and used for subsequent rounds of selection.

After the third round of selection, eluted phages were cloned, propagated, and sequenced to determine the nucleotide sequence encoding the foreign peptide.

TABLE 4

Foreign peptides expressed by phage selected
for ability to bind Salmonella in solution

| | | |
|---|---|---|
| Peptide sequence | Frequency | SEQ ID NO: |

| | | |
|---|---|---|
| DRSPSSPT | 4 | 40 |
| VPIPYNGE | 1 | 41 |
| VSSNQAPP | 18 | 42 |

### Example 4

### Evaluation of Binding Ability of Phase

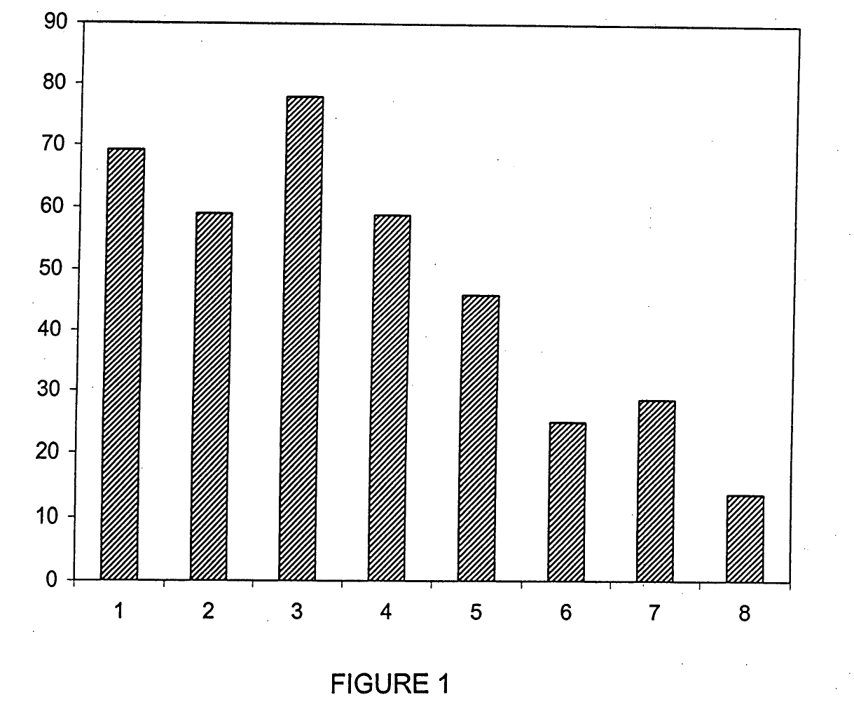

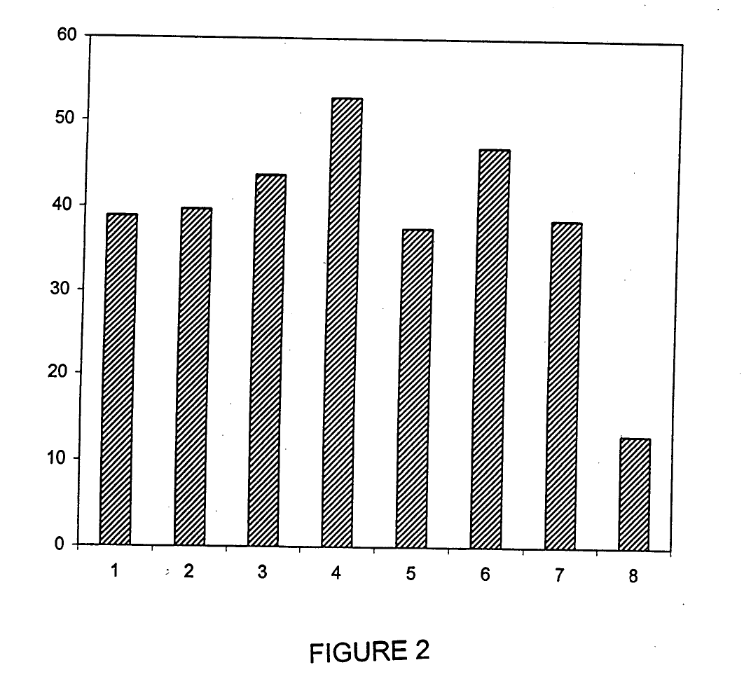

Phage clones displaying foreign peptides representing various sequences were screened with two ELISA formats for those exhibiting the best binding properties. In one ELISA format, phage were evaluated for their ability to bind to Salmonella adsorbed to a plastic Petri dish (results shown in FIG. 1). In another ELISA format, phage were adsorbed to plastic and evaluated by the binding of Salmonella to the adsorbed phage (results shown in FIG. 2).

In another evaluation, phage bearing selected foreign peptides were evaluated using a co-precipitation test which was similar to the selection scheme used in Example 3. Results of this co-precipitation test are shown in FIG. 3. The phage exhibiting the best binding in this test demonstrated binding to Salmonella cells which was 12,000-22,000 times higher than the level exhibited by the control vector phage f8-5.

### Example 5

### Fluorescent Phage Probes for Detection of S. typhimurium

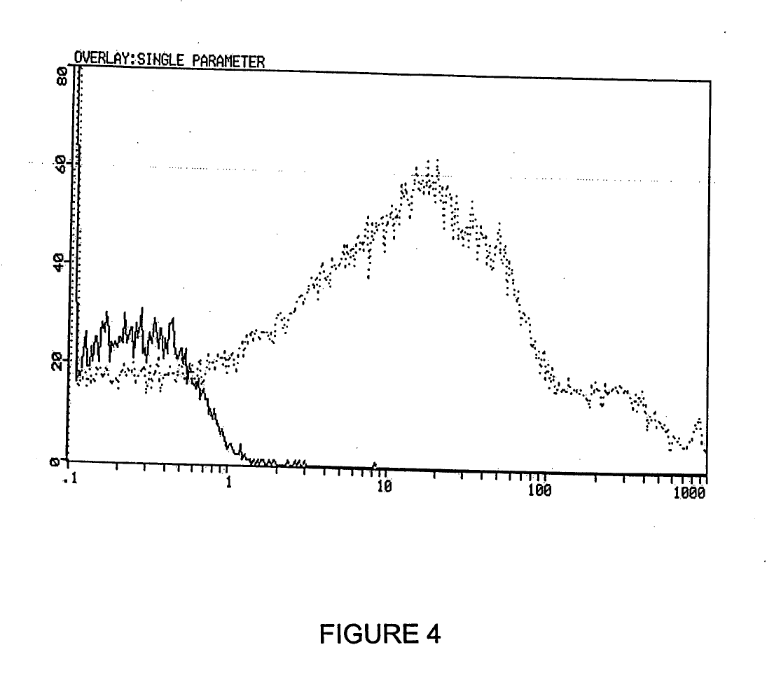

Salmonella were mixed with fluorescently-labeled phage and incubated for 1 hour. Unbound phage was removed by washing with TBS/0.5% Tween™. Salmonella cells were then analyzed by Fluorescence-Activated Cell Sorting (FACS) and fluorescent microscopy. As shown in FIG. 4, FACS analysis revealed that phage bound to most Salmonella cells. This result was supported by results of fluorescence microscopy of the Salmonella cells showing that the fluorescent phage had bound to the cells.

### Example 6

### Ligand Sensor Device

Synthetic peptides of the invention are used to create a ligand sensor device (LSD) as described in copending application Ser. No. 10/068,570, filed Feb. 6, 2002. Briefly, synthetic peptides comprising the sequence DLTSNQAT are coupled to a ligand sensor device.

The coupling composition layer of the LSD is the layer which couples the peptide of interest to the sensor. In some embodiments, this coupling composition layer is composed of streptavidin and biotin. Any composition or coupling method may be used so long as the peptide of interest is coupled to the sensor and the LSD is capable of detecting the binding of a ligand to the peptide of interest. Thus, in some embodiments, a sensor is coated with gold or a gold-coated sensor is obtained; the sensor is then coated with streptavidin and coupled to the biotinylated peptide of interest. Alternatively, the coupling composition layer may comprise a biotinylated thiol or disulfide layer which is linked directly to a layer of gold; the biotinylated layer is then linked to streptavidin and the biotinylated peptide of interest. See, for example, Luppa et al. (2001) Clinica Chimica Acta 314: 1-26; Gau et al. (2001) Biosensors & Bioelectronics 16: 745-755.

The peptide of interest may optionally be combined with a spacer to enhance the performance of the LSD. Spacers may be short peptides which are synthesized in continuous linkage with the peptide of interest to create a combination of the peptide of interest and the spacer; this combination may then be biotinylated or chemically modified in order to couple it to the sensor. One of skill will recognize that the length of the spacer may affect the efficacy of the LSD: if the spacer is too short, the ligand will not have sufficient access to the peptide and binding will be decreased; if the spacer is too long, the orientation of the peptide may be disadvantageously altered. A peptide spacer may be 1, 2, 3, 4, 5, 6, 7, 8, 9, 10, 11, 12, 15, 20, 25, 30, or more amino acids in length. One of skill can adjust the length of the spacer to optimize the efficacy and sensitivity of the LSD.

The LSD includes a piezoelectric crystal sensor. In some embodiments, an acoustic wave sensor is used which comprises an AT-cut planar quartz crystal with a 5 MHz nominal oscillating frequency. Such crystals, suitable for acoustic wave devices (AWD), are commercially available (e.g., Maxtek, Inc). The crystals or sensors may be supplied with electrodes, for example, crystals may be supplied with circular gold electrodes deposited on both sides of the crystal for the electrical connection to the oscillatory circuit. In some embodiments, a mass-sensitive sensor is used; alternatively, other sensors may be used so long as they are capable of detecting the binding of peptide of interest to ligand and providing signal output that changes in response to that binding. A direct correlation of binding and signal output is not required so long as the desired result is obtained. Thus, when binding occurs, different physical and electrochemical properties of the sensor may be changed: mass; free energy; electrical properties such as charge and conductance; optical properties such as fluorescence, luminescence, adsorption, scatter, and refraction. Accordingly, suitable sensors include electrochemical, calorimetric, and optical sensors. See, for example, Luppa et al. (2001) Clinica Chimica Acta 314: 1-26. One of skill in the art will appreciate that for different applications of the assays of the invention, sensors with different sensitivities and outputs may be used. Thus, for example, in some applications a preferred LSD will be capable of high-resolution quantitation of changes in binding, while for other applications an LSD need only detect the presence or absence of high-affinity binding.

In some embodiments, a Maxtek 740 sensor is used which has a working frequency of 5 MHz. One of skill recognizes that the working frequency corresponding to the highest sensitivity of the LSD system can be identified to optimize the changes in the resonance frequency of the sensor when ligand is bound. Any suitable device may be used to monitor the signal output from the sensor; for example, an HP4195A Network/Spectrum Analyzer (Hewlett-Packard) can be used. The analyzer device scans a set range of frequencies and measures the signal properties at each frequency. After the optimal frequency is found for a particular peptide/ligand combination this frequency can be used as a working frequency for sensitive measurements of binding; useful frequencies are generally be Tween™ 2 MHz and 150 MHz.

The sensor is prepared to be coupled to the peptide by any suitable method. For example, a Langmuir-Blodgett film of biotinylated lipid is added to the sensor, as further described below. Langmuir-Blodgett films are formed from at least one monolayer. A monolayer is a one molecule thick film of at least one amphiphilic compound or composition that forms at the interface of an aqueous solution with the ambient air. Each molecule in the monolayer is aligned in the same orientation, with the hydrophobic domain facing the air and the hydrophilic domain facing the aqueous solution. Compression of the monolayer results in the formation of an ordered, two-dimensional solid that may be transferred to a substrate by passing the substrate through the monolayer at the air/water interface. A monolayer that has been transferred to a substrate is termed a Langmuir-Blodgett film, or LB film. For reviews of Langmuir-Blodgett technology, see Gaines, G. L. Jr. (1966) Insoluble Monolayers at Liquid-Gas Interfaces, Interscience, New York; Zasadzinski et al. (1994) Science 263:1726-1733; Ullman (1991) An Introduction to Ultrathin Organic Films, Academic Press, Boston, Mass.; and Roberts (1990) Langmuir-Blodgett Films, Plenum, New York; the contents of which are incorporated herein by reference.

Monolayers are typically composed of organic molecules such as lipids, fatty acids and fatty acid derivatives, fat soluble vitamins, cholesterol, chlorophyll, valinomycin and synthetic polymers such as polyvinyl acetate and polymethyl methacrylate. Monolayers may also be formed by many other amphiphilic compounds; thus, many amphiphilic compounds may be used to form the monolayers of the invention. Such compounds include lipids having at least 14 carbon atoms. Examples include stearic acid and hexadecanoic acid. Other compounds that will form monolayers include, but are not limited to those described in Gaines, G. L. Jr. (1966) Insoluble Monolayers Liquid-Gas Interface, Interscience, New York, the contents of which are incorporated by reference.

Lipid monolayer depositions may be carried out by methods known in the art and as described in copending application Ser. No. 09/452,968, filed Dec. 2, 1999, herein incorporated by reference in its entirety. Langmuir-Blodgett (LB) film balances are commercially available, for example from KSV-Chemicals, Finland, and are operated in accordance with the supplier's instructions.

The Langmuir-Blodgett film is formed by the successive transfer of monolayers onto the surface of the sensor using the Langmuir-Blodgett technique. In some embodiments, biotinylated lipid solutions are spread on the aqueous subphase as hexane solutions. The monolayer is then compressed and a vertical film deposition is performed. In LB film deposition, multiple monolayers may be added to the sensor by successive dipping of the sensors through the monomolecular film deposited at the air/liquid interface. LB films may be formed by the addition of one, two three, four, five, six, seven, eight, nine, ten, eleven, twelve, thirteen, fourteen, fifteen or more monolayers in this manner to create the final Langmuir-Blodgett film.

The monolayers used to create the Langmuir-Blodgett film may be formed without the aid of a volatile organic solvent. See, for example, copending application Ser. No. 09/452,968, filed Dec. 2, 1999, hereby incorporated by reference in its entirety. In some embodiments, a peptide of interest is covalently bound or linked to phospholipids; vesicles comprising these phospholipids are then used to create monolayers and LB films to make an LSD of the invention. In such embodiments, the coupling of the peptide of interest to the sensor may be accomplished by the formation of such an LB film on the sensors and does not necessarily require a coupling via streptavidin and biotin interactions. Such sensors may be gold-plated or coated with other material to facilitate the adherence of the LB film to the sensor.

Generally, the formation of a monolayer without the aid of an organic solvent is formed by layering an amphiphilic compound or composition onto an aqueous subphase by slowly allowing this compound or composition to run down an inclined wettable planar surface that is partially submersed into the subphase. The formation of a monolayer in this way comprises the steps of:
(a) providing a composition comprising at least one amphiphilic compound, wherein said composition contains not more than 25% of a volatile organic solvent and may optionally contain one or more compounds of interest;
(b) immersing one end of a wettable planar surface into an aqueous subphase, wherein said planar surface forms an angle of about 90-170 degrees to an upper surface of said subphase, wherein said subphase comprises at least one monovalent cation and at least one bivalent cation, wherein said subphase has a pH of 4.0-8.0;
(c) delivering said composition at a rate of about 0.02-4.0 ml per minute to said planar surface to form a monolayer; and
(d) compressing said monolayer.

After the desired surface pressure is achieved by compression of the monolayer, an LB film may be formed by passing a substrate through the monolayer one or more times. Methods of forming LB films are known to those skilled in the art and are described in Ullnan (1991) An Introduction to Ultrathin Organic Films, Academic Press, Boston, Mass.; and Roberts (1990) Langmuir-Blodgett Films, Plenum, New York; the contents of which are incorporated herein by reference. Once the LB film is formed, the peptide of interest may be coupled to the LB film.

Once the LSD is prepared, the signal output may be measured by any suitable device which is compatible with the crystal or sensor used to create the LSD. Many such devices are known in the art and are commercially available. In some embodiments, measurements are carried out using a PM-740 Maxtek plating monitor with a frequency resolution of 0.5 Hz at 5MGz. By “signal output” is intended any property of the sensor that changes in response to binding of a ligand and can be detected or monitored by a suitable device. Signal output of the device may be recorded and analyzed using a personal computer and appropriate data acquisition card and software. In some embodiments, the resonance frequency varies with the mass of the crystal as it changes due to interaction of ligands with the sensor. Because the voltage output from the Maxtek device is directly related to the resonance frequency of the quartz crystal sensor, changes in the resonance frequency and/or voltage may then be used to monitor the binding of ligand to the peptide of interest. The change in frequency and voltage will be proportional to the concentration of ligand, provided that nonspecific binding is low. Once prepared, an LSD may be used for multiple assays and may remain functional for a long period of time, up to a day, several days, a week, or a month or more.

The LSD is exposed to one or more ligands, typically by layering a solution of homogenate of the tissue or cell type of interest onto the LSD, although cell suspensions or other types of cell or tissue preparations may also be used. For other applications, solutions of purified or somewhat purified ligands may be exposed to the LSD. Thus, any sample may be assayed for the presence of ligands by exposure to an LSD, so long as the form of the sample is compatible with exposure to the LSD.

### Example 7

### Phase Ligand Sensor Device

Synthetic peptides of the invention are used to create a phage ligand sensor device (PLSD) as described in copending application Ser. No. 10/289,725, filed Nov. 7, 2002. Briefly, phage are engineered to express the synthetic peptide DRPSPNTV as a fusion protein with the phage coat protein pVIII. An aliquot of phage is biotinylated using commercially available reagents and standard procedures (see, e.g., 2002 catalog from Pierce Biotechnology, Inc., Rockford, Ill.).

To prepare the sensor component of the device, AT-cut planar quartz crystals with a 5 MHz nominal oscillating frequency are obtained (Maxtek, Inc.). Circular gold electrodes are deposited on both sides of the crystal sensor for the electrical connection to the oscillatory circuit. The microbalance and sensor are calibrated by the deposition of well characterized stearic acid monolayers. The deposition of an increasing number of stearic acid monolayers on the surface of a sensor results in a linear increase in mass.

To prepare the sensor to be coupled to the phage, monolayers containing phospholipid (N (biotinoyl)-1,2-dihexadecanoyl-sn-glycero-3-phosphoethanolamine) are transferred onto the gold surface of an acoustic wave sensor using the Langmuir-Blodgett technique to create a biotinylated sensor surface. Multilayers are obtained by successive dipping of the sensor through the monomolecular film deposited at a water-air interface. The phage is coupled with the phospholipid via streptavidin intermediates by molecular self-assembly to create a PLSD.

Lipid monolayer depositions are carried out using a Langmuir-Blodgett (LB) film balance KSV 2200 LB (KSV-Chemicals, Finland). This fully computerized system contains a Wilhelmy-type surface balance (range 0-100 mN/m; sensitivity 0.05 mN/m), a Teflon trough (45×15 cm²), a variable speed motor-driven Teflon barrier (0-200 mm/min), and a laminar flow hood. The trough is mounted on a 200 kg marble table. Vibration control is provided by interposing rubber shock absorbers, and by mounting the laminar flow hood on a separate bench. Surface pressure is monitored by use of a sandblasted platinum plate of 4 cm perimeter. The temperature of the aqueous subphase (20° C.±0.1° C.) is measured by a thermistor located just below the air/liquid interface and controlled by water circulation through a quartz tube coil on the bottom of the trough.

Lipid solutions are spread on the aqueous subphase as hexane solutions (1 mg/ml) containing 2% ethanol (Ito et al. (1989) Thin Solid Films 180: 1-13). The aqueous subphase used in the experiments is a solution containing 55 mM KCl, 4 mM NaCl, 0.1 mM CaCl₂, 1 mM MgCl₂ and 2 mM 3-(N-morpholino)-propanesulfonic acid (MOPS) made with deionized double distilled water (pH 7.4). After spreading, the monolayer is allowed to equilibrate and stabilize for 10 min at 19° C. The monolayer is then compressed at a rate of 30 mm/min and a vertical film deposition is carried out with a vertical rate of 4.5 mm/min and at a constant surface pressure of 25 mN/m. Eleven monolayers are transferred to the gold surface of the quartz crystals in this manner. Monolayers and multilayers deposited by LB technique are reasonably stable (see Pathirana et al. (2000) Biosensors & Bioelectronics 15: 135-141).

The PLSD is then assembled using the “molecular assembly” of biotin/streptavidin coupling. Streptavidin is added to immobilize the biotinylated phage on the sensor (covered with biotinylated lipids) as follows. The sensor is treated with subphase solution containing 0.01 mg/ml streptavidin for 2 hours, then rinsed with distilled water and dried for 2 minutes in ambient air. The sensor is then exposed to subphase solution containing biotinylated phage at 0.001 mg/ml for 2 hours and then rinsed and dried again as above. If necessary, these steps can be followed by a blocking step with a subphase solution containing biotin to prevent nonspecific binding of naturally biotinylated proteins to the sensor. Each prepared PLSD may then be placed in an individual Petri dish and stored at 4° C. Tests are generally performed within 24 hours of assembling the PLSD.

Binding measurements are carried out using a PM-700 Maxtek plating monitor with a frequency resolution of 0.5 Hz at 5 MHz. Voltage output of the Maxtek device can be recorded and records are analyzed offline. The voltage output from the Maxtek device is directly related to the resonance frequency of the quartz crystal sensor. Changes in the resonance frequency of the quartz crystal sensor are used to monitor the binding of Salmonella to the sensor surface. For binding measurements, the PLSD is positioned in the probe arm of the instrument just before delivery of samples. After recording is started, 1000 μl PBS is delivered with a pipette to the PLSD surface and voltage is recorded for 4-8 minutes. Then the PBS is removed carefully with a plastic pipette tip and a new recording is initiated. Different solutions containing Salmonella are added sequentially to the sensor and the same measuring procedure is followed after each addition. Each experiment is replicated 2-4 times, and the temperature of all samples is approximately 25° C. The data collected may be stored and analyzed offline. The ratio of occupied (Y) and free (1-Y) phages on the sensor surface can be determined as
log(Y/(1−Y))=log K_{b}+nlog[C] [1]
where K_{b} is the association binding constant, C is a Salmonella concentration, and n is the number of molecules bound to a single phage. A plot of the left-hand side of equation (1) versus log[C] is known as a Hill plot (see Kuchel & Ralston (1988) Theory and Problems of Biochemistry (McGraw-Hill, New York)). A Hill plot gives an estimate of n from the slope, K_{b} from the ordinate intercept, and EC₅₀ at the point when Y=1−Y. For each Salmonella concentration, the sensor signal approaches a steady-state value corresponding to that concentration. The response curves are distinguished by the fast reaction time, the attainment of a steady state, and low non-specific binding.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A synthetic peptide, wherein the amino acid sequence of said peptide comprises a sequence containing the motif V (P/S/T) P (N/P/Q) (P/Q/S/T) (A/N/Q/S) (H/P/S) (A/P/Q/S).

2. A pharmaceutical composition comprising the synthetic peptide of claim 1.

3. The pharmaceutical composition of claim 2, wherein said synthetic peptide is conjugated to a compound.

4. The pharmaceutical composition of claim 3, wherein said compound is selected from the group consisting of antibiotics.

5. The synthetic peptide of claim 1, wherein said peptide is eight amino acids in length.

6. A nucleotide sequence encoding the synthetic peptide of claim 1.

7. A vector comprising the nucleotide sequence of claim 6.

8. A phage clone expressing the synthetic peptide of claim 1.

9. A ligand sensor device comprising the synthetic peptide of claim 1.

10. A synthetic peptide, wherein the amino acid sequence of said peptide comprises a sequence containing the motif D P (H/K/R) (G/L/P/S) (A/P) (A/G/H/L/Q) (G/H/Q/S) (L/M/T).

11. A pharmaceutical composition comprising the synthetic peptide of claim 10.

12. The pharmaceutical composition of claim 11, wherein said synthetic peptide is conjugated to a compound selected from the group consisting of antibiotics.

13. The synthetic peptide of claim 10, wherein said peptide is eight amino acids in length.

14. A nucleotide sequence encoding the synthetic peptide of claim 10.

15. A vector comprising the nucleotide sequence of claim 14.

16. A phage clone expressing the synthetic peptide of claim 10.

17. A synthetic peptide, wherein the amino acid sequence of said peptide comprises a sequence containing the motif (D/E) R(P/S/T) (P/S/T) (P/S) (A/N/S) (H/P/T) (T/V).

18. A nucleotide sequence encoding the synthetic peptide of claim 17.

19. A pharmaceutical composition comprising the synthetic peptide of claim 17.

20. A synthetic peptide, wherein the amino acid sequence of said peptide comprises the sequence DLTSNQAT.

21. A phage ligand sensor device comprising a synthetic peptide having the amino acid sequence VTPPTQHQ.
